(19) United States
(12) Patent Application Publication
(10) Pub. No.: US 2025/0249087 A1
(43) Pub. Date: Aug. 7, 2025

(54) CONSTRUCTION OF NOVEL HUMAN INFLUENZA VIRUS VACCINE AND APPLICATION

(71) Applicant: Wuhan JinYiTai Biological Co., Ltd., Wuhan (CN)

(72) Inventors: Yeping TAN, Wuhan (CN); Haiwang ZOU, Wuhan (CN); Hui ZHENG, Wuhan (CN); Huashan ZHOU, Wuhan (CN)

(73) Assignee: Wuhan JinYiTai Biological Co., Ltd., Wuhan (CN)

(21) Appl. No.: 19/002,606

(22) Filed: Dec. 26, 2024

(30) Foreign Application Priority Data
Feb. 2, 2024 (CN) 202410145547.4

(51) Int. Cl. A61K 39/145; A61K 39/00; A61K 40/11; A61K 40/31; A61K 40/42; A61P 35/00; A61P 37/04; C12N 7/00; C12N 15/86

(52) U.S. Cl. CPC A61K 39/145; A61K 40/11; A61K 40/31; A61K 40/42; A61P 35/00; A61P 37/04; C12N 7/00; C12N 15/86; A61K 2039/5256; A61K 2039/585; C12N 2710/10343; C12N 2760/16122; C12N 2760/16134; C12N 2760/16171

(57) ABSTRACT

The present disclosure discloses a method and an application of a human influenza virus vaccine The method and a non-replicating recombinant adenovirus named Ad-JYT-CMV-recHA promoter to control E1B19K or E1B19K-E1B55K fusion protein gene, and recombinant oncolytic adenoviruses named Ad-EE1A-hTERT-recHA and Ad-HTE1A-hTERT-recHA, respectively is constructed by optimizing a nucleotide sequence for influenza virus hemagglutinin HA, and thus the recHA gene is overexpressed in mammalian cells and the recHA is anchored on the cell surface and used for preventing influenza virus cold; human TERT promoter is used to control recHA gene, and a novel influenza recHA vaccine is introduced into an oncolytic adenovirus therapy as a tumor-heterologous artificial antigen for treating a tumor; furthermore, the recHA glycoprotein is anchored on the tumor cell surface as a tumor-specific artificial target, which is expected to combine with a CART-HA cell for treating a solid tumor.

### RELATED APPLICATIONS

The present application claims the benefit of priority of Chinese Patent Application No. 202410145547.4, filed 2 Feb. 2024, and entitled “CONSTRUCTION OF NOVEL HUMAN INFLUENZA VIRUS VACCINE AND APPLICATION,” the entire contents of each of which are incorporated herein by reference.

### TECHNICAL FIELD

The present disclosure relates to the technical field of virus vaccines and in particular to a construction method and an application of a novel human influenza virus vaccine.

### BACKGROUND ART

Influenza viruses are classified into three types: A, B, and C. Influenza viruses discovered in recent years will be classified as type D. Influenza viruses may cause infections and diseases in various animals such as humans, birds, pigs, horses, and bats, and are pathogens responsible for human and animal diseases such as human influenza, avian influenza, swine influenza and equine influenza.

Human influenza is mainly caused by influenza A virus and influenza B virus. There are mainly two glycoprotein antigens, hemagglutinin (HA) and neuraminidase (NA), on the surface of influenza viruses. HA is divided into two parts, H1 and H2. Due to the instability of the single-stranded negative-sense RNAs of the H1 region and NA, antigenic variations frequently occur, leading to viral influenza outbreaks. Influenza A virus has been coexisting with human beings for more than 100 years since the global pandemic in 1918. Influenza A viruses can be further divided into subtypes such as H1N1, H3N2, H5N1, H7N9 and H9N2. The main reason for the recurring epidemics of influenza viruses is antigenic variations in the surface glycoproteins HA and NA. A pattern in which influenza virus HA and NA undergo minor variations each year, leading to localized outbreaks, and major variations every ten years, resulting in widespread or even global pandemics, has been basically established, posing a threat to human health.

The primary method for human beings to prevent influenza virus infection is vaccination. Currently, in many countries around the world, chicken embryos and MDCK cells are mainly used to propagate influenza viruses, and after purification, these viruses are prepared into inactivated whole virus vaccines, split virus vaccines, and subunit vaccines. Only in the United States of America, there is a human influenza virus protein vaccine produced with a baculovirus expression system. Novel coronavirus mRNA vaccines and adenovirus vector vaccines are both produced by expressing the whole genes of the surface glycoproteins and these surface glycoproteins are anchored on the cell surface.

The whole gene for the wild-type human H1N1 influenza virus HA glycoprotein cannot be artificially expressed in mammalian cells due to hydrophobic amino acid enrichment in the transmembrane region; therefore, there is currently no influenza virus vaccine in the world like a novel coronavirus surface glycoprotein that is expressed in the human body and anchored on the cell surface.

There are about 20 million new cancer patients and about 10 million deaths worldwide every year. Up to now, although new anti-cancer drugs or methods, such as monoclonal antibodies, small molecule targeted drug, CART cell and oncolytic viruses, are constantly coming out. The treatment of tumors, especially solid tumors, is still dominated by surgery, chemotherapy and radiotherapy, and no satisfactory results have been achieved. Tumors are still a serious threat to human health. It is still a challenge for scientists to develop new anti-tumor drugs and find new methods to treat tumors.

### SUMMARY OF THE INVENTION

An object of the present disclosure is to provide a construction method and an application of a novel human influenza virus vaccine, so as to solve the above shortcomings in the prior art.

In order to achieve the above object, the present disclosure provides the following technical solutions: A method for constructing a novel human influenza virus vaccine is provided, which specifically comprises the following steps:
S1. constructing recombinant HA (recHA) using a human influenza virus H1N1 hemagglutinin glycoprotein gene, wherein the influenza virus hemagglutinin glycoprotein gene comprises amino acid sequences and optimized nucleotide sequences of HA1 and HA2, the recHA is constructed by replacing a transmembrane region and an intracellular sequence of a wild-type HA gene with a transmembrane region of human epidermal growth factor receptor-2 (HER-2), and the human influenza virus is vaccine strain H1N1 NYMC X-179A recommended by the World Health Organization in which hemagglutinin glycoprotein acts as an antigen and includes HA glycoproteins of all human influenza virus subtypes;
S2. synthesizing a hamster Enhancer TERT DNA fragment, the 5′ end and 3′ end of which are provided with restriction sites Xbal and EcoRI respectively, digesting shuttle vector pDC316 and the hamster Enhancer TERT DNA with double enzymes, Xbal and EcoRI, respectively, followed by recovery, and inserting the synthesized Hamster Enhancer TERT DNA fragment at the Xbal and EcoRI sites for enzymatic linkage to construct a vector named pDC316-hamsterTERT;
S3. synthesizing an EF1a DNA fragment, the 5′ end and 3′ end of which are provided with restriction sites Xbal and EcoRI respectively, digesting shuttle vector pDC316 and EF1a DNA with double enzymes, Xbal and EcoRI, respectively, followed by recovery, and inserting the synthesized EF1a DNA fragment at the Xbal and EcoRI sites for enzymatic linkage to construct a vector named pDC316-EF1a;
S4. synthesizing EIA gene, the 5′ end and 3′ end of which are provided with restriction sites EcoRI and BgIII respectively, digesting vector pDC316-hamsterTERT and the E1A gene with double enzymes, EcoRI and BgIII, respectively, followed by recovery and linkage to construct a vector named pDC316-hamsterTERT-E1A, subjecting the ELA gene and vector pDC316-EF1a to PCR, recovering the PCR products respectively, performing seamless cloning to construct a vector named pDC316-EF1a-E1A, and identifying the correctness of DNA insertion into the recombinant vector by sequencing;
S5. synthesizing a Survivin fragment, the 5′ end and 3′ end of which are provided with restriction sites Xbal and EcoRI respectively, digesting shuttle vector pDC316 and Survivin with double enzymes, Xbal and EcoRI, respectively, followed by recovery, and inserting the synthesized Survivin fragment at the Xbal and EcoRI sites for enzymatic linkage to construct a vector named pDC316-Survivin;
S6. synthesizing E1B19K-2A-E1B55K gene, the 5′ end and 3′ end of which are provided with restriction sites EcoRI and Sall respectively, digesting vector pDC316-Survivin and the E1B19K-2A-E1B55 gene with double enzymes, EcoRI and Sall, respectively, followed by recovery, and inserting the synthesized E1B19K-2A-E1B55K gene at the EcoRI and Sall sites for enzymatic linkage to construct a vector named pDC316-Survivin-E1B19K-2A-E1B55K; and
with the vector pDC316-Survivin-E1B19K-2A-E1B55K as a template, amplifying the genes Survivin-E1B19K and Survivin-E1B19K-2A-E1B55K respectively, and inserting the amplified products into the vectors pDC316-EF1a-E1A and pDC316-hamsterTERT-E1A respectively by seamless cloning, to construct vectors named pDC316-EF1a-E1A-Survivin-E1B19K and pDC316-hamsterTERT-E1A-Survivin-E1B19K-2A-E1B55K, respectively;
S7. synthesizing the hTERT-recHA DNA, subjecting the hTERT-recHA DNA to PCR and inserting the PCR product into the vectors pDC316-EF1a-E1A-Survivin-E1B19K and pDC316-hamsterTERT-E1A-Survivin-E1B19K-2A-E1B55K by seamless cloning, to construct vectors named pDC316-EF1a-E1A-Survivin-E1B19K-hTERT-recH and pDC316-hamsterTERT-E1A-Survivin-E1B19K-2A-E1B55K-hTERT-recHA;
S8. PCR cloning a recHA DNA and inserting the recHA DNA downstream of CMV promoter in pDC316 vector to construct a non-replicating adenovirus recombinant vector named pDC316-CMV-recHA;
S9. co-transfecting the pDC316-CMV-recHA recombinant vector and a backbone plasmid into HEK293 cells (Low passage) to obtain a non-replicating recombinant adenovirus named Ad-JYT-CMV-recHA;
S10. co-transfecting the recombinant vectors pDC316-EF1a-E1A-Survivin-E1B19K-hTERT-recH and pDC316-hamsterTERT-E1A-Survivin-E1B19K-2A-E1B55K-hTERT-recHA respectively, and a backbone plasmid, into HEK293 cells to obtain recombinant adenoviruses named Ad-EE1A-hTERT-recHA and Ad-HTE1A-hTERT-recHA, respectively; and
S11. subjecting the recombinant adenoviruses in steps S9 and S10 to plaque purification, then adding the purified recombinant adenoviruses to cultured HEK293 cells and 293Low passage cells, collecting 50 ml of a culture solution and cells after 72 hours, and taking a supernatant for later use; and adding a buffer solution to the cells, performing ultrasonic disruption, removing cell debris, then mixing the remaining material with the supernatant, adding 40% PEG 8000 and NaCl for pelleting, collecting a pellet, then adding a buffer solution to prepare a suspension, performing chromatography using molecular sieve Sepharose 4FF, followed by ultrafiltration, centrifugation and concentration to obtain a purified recombinant adenovirus, and storing the purified recombinant adenovirus in a refrigerator at −80° C. for later use.

A recombinant virus strain is provided, which is constructed by the above method.

The recombinant virus strain allows HA to be a tumor-specific artificial target, which is used in combination with a CART-HA cell for treating a tumor.

The constructed recombinant virus strain can be used for preparing a novel human influenza virus vaccine.

A novel human influenza virus vaccine is provided, wherein an antigen in the novel human influenza virus vaccine comprises the above recombinant virus strain.

Use of the novel human influenza virus vaccine in the field of treatment of influenza virus cold and tumors is provided.

Compared with the prior art, the construction method and the application of a novel human influenza virus vaccine, as provided by the present disclosure, solve the technical problem that the whole gene of human influenza virus wild-type H1N1 subtype HA glycoprotein cannot be expressed in mammalian cells. The present inventors not only develop a novel human influenza virus vaccine, but also pave the way for the research and development of human influenza virus mRNA vaccines, making it possible for human beings to use more advanced influenza virus vaccines to prevent influenza virus infection.

In order to treat a tumor, by introducing a novel influenza recHA vaccine into an oncolytic adenovirus, an Ad-EE1A-hTERT-recHA vector is constructed, wherein EF1a promoter is used to control E1A gene, Survivin promoter is used to control E1B19K gene, and E1B55K gene is deleted, thereby improving the oncolytic ability and safety of the oncolytic adenovirus. An Ad-HTE1A-hTERT-recHA vector is constructed, wherein hamster TERT promoter is used to control E1A gene, and Survivin promoter is used to control E1B19K and E1B55K fusion protein gene, thereby realizing selective replication of the oncolytic adenovirus in a specific tumor. Human TERT promoter is used to control recHA gene, thereby enabling the recHA gene to be expressed in a specific tumor and the recHA to be anchored on the tumor cell surface as a tumor-heterologous artificial antigen and a tumor-heterologous artificial target.

Anti-HA antibodies and memory B cells that have been produced in vivo are utilized. Before Clinical treatment, a patient is injected with an influenza virus HA vaccine. When anti-HA antibodies exist in the patient, the recombinant oncolytic adenovirus carrying influenza virus hemagglutinin gene (recHA) is then injected. Once the hemagglutinin protein is expressed in the tumor and anchored on the tumor cell surface, the immune system of the body is induced to accurately identify and eliminate the tumor cells; furthermore, the recHA glycoprotein is anchored on the tumor cell surface, and the HA acts as a tumor-specific artificial target and is expected to combine with a CART-HA cell for treating a solid tumor.

### BRIEF DESCRIPTION OF THE DRAWINGS

In order to more clearly illustrate the examples of the present application or the technical solutions in the prior art, the accompanying drawings needed in the examples will be briefly described below. Obviously, the accompanying drawings in the following description are only some examples disclosed in the present disclosure, and other accompanying drawings may also be obtained by those of ordinary skill in the art according to these accompanying drawings.

FIG. 1 shows a schematic diagram of the construction of recombinant adenovirus vectors Ad-JYT-CMV-recHA, Ad-EE1A-hTERT-recHA and Ad-HTE1A-hTERT-recHA as provided by an example of the present disclosure.

FIG. 2 shows a first schematic diagram of the results of a Western blot assay as provided by an example of the present disclosure.

FIG. 3 shows a second schematic diagram of the results of a Western blot assay as provided by the example of the present disclosure.

Figures for 4A and 4B show schematic diagrams of the results of a flow cytometry assay as provided by an example of the present disclosure.

FIG. 5 shows a schematic diagram of the results of anti-HA IgG in female mice as provided by an example of the present disclosure.

FIG. 6 shows a schematic diagram of the results of serum anti-HA IgG in Syrian hamsters as provided by an example of the present disclosure.

FIG. 7 shows a first schematic diagram of the results of a tumor-killing experiment in Syrian hamsters as provided by an example of the present disclosure.

FIG. 8 shows a second schematic diagram of the results of a tumor-killing experiment in Syrian hamsters as provided by the example of the present disclosure.

FIG. 9 shows a schematic diagram of the results of a tumor cells re-inoculation experiment in cured Syrian hamsters as provided by an example of the present disclosure.

### DETAILED DESCRIPTION OF EMBODIMENTS

In order to make the technical solutions of the present disclosure better understood by those skilled in the art, the present disclosure will be further described below in detail in conjunction with the accompanying drawings.

### Example 1

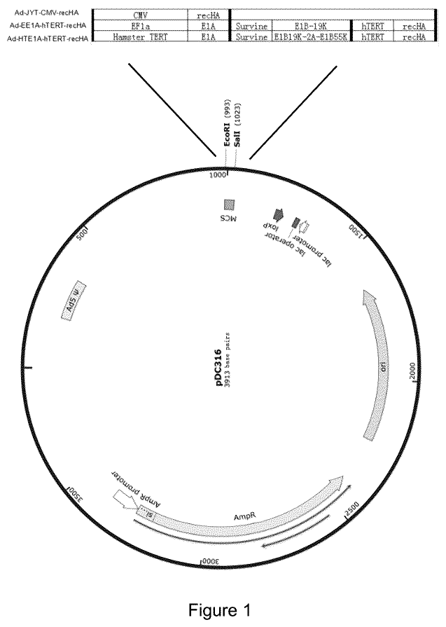

Referring to FIG. 1, a method for constructing a novel human influenza virus vaccine is provided, which specifically comprises the following steps:
S1. constructing recombinant HA (recHA) using a human influenza virus H1N1 hemagglutinin glycoprotein gene, wherein the influenza virus hemagglutinin glycoprotein gene comprises amino acid sequences and optimized nucleotide sequences of HA1 and HA2, the recombinant HA (recHA) is constructed by replacing a transmembrane region and an intracellular sequence of a wild-type HA gene with a transmembrane region of human epidermal growth factor receptor-2 (HER-2), and the human influenza virus is vaccine strain H1N1 NYMC X-179A recommended by WHO in which hemagglutinin glycoprotein acts as an antigen and includes HA glycoproteins of all human influenza virus subtypes;
S2. synthesizing a hamster Enhancer TERT DNA fragment, the 5′ end and 3′ end of which are provided with restriction sites Xbal and EcoRI respectively, digesting shuttle vector pDC316 and the hamster Enhancer TERT DNA with double enzymes, Xbal and EcoRI, respectively, followed by recovery, and inserting the synthesized Hamster Enhancer TERT DNA fragment at the Xbal and EcoRI sites for enzymatic linkage to construct a vector named pDC316-hamsterTERT;
S3. synthesizing an EF1a DNA fragment, the 5′ end and 3′ end of which are provided with restriction sites Xbal and EcoRI respectively, digesting shuttle vector pDC316 and EF1a DNA with double enzymes, Xbal and EcoRI, respectively, followed by recovery, and inserting the synthesized EF1a DNA fragment at the Xbal and EcoRI sites for enzymatic linkage to construct a vector named pDC316-EF1a;
S4. synthesizing EIA gene, the 5′ end and 3′ end of which are provided with restriction sites EcoRI and BgIII respectively, digesting vector pDC316-hamsterTERT and the E1A gene with double enzymes, EcoRI and BgIII, respectively, followed by recovery and linkage to construct a vector named pDC316-hamsterTERT-E1A, subjecting the EIA gene and vector pDC316-EF1a to PCR, recovering the PCR products respectively, performing seamless cloning to construct a vector named pDC316-EF1a-E1A, and identifying the correctness of DNA insertion into the recombinant vector by sequencing;
S5. synthesizing a Survivin fragment, the 5′ end and 3′ end of which are provided with restriction sites Xbal and EcoRI respectively, digesting shuttle vector pDC316 and Survivin with double enzymes, Xbal and EcoRI, respectively, followed by recovery, and inserting the synthesized Survivin fragment at the Xbal and EcoRI sites for enzymatic linkage to construct a vector named pDC316-Survivin;
S6. synthesizing E1B19K-2A-E1B55K gene, the 5′ end and 3′ end of which are provided with restriction sites EcoRI and Sall respectively, digesting vector pDC316-Survivin and the E1B19K-2A-E1B55 gene with double enzymes, EcoRI and Sall, respectively, followed by recovery, and inserting the synthesized E1B19K-2A-E1B55K gene at the EcoRI and Sall sites for enzymatic linkage to construct a vector named pDC316-Survivin-E1B19K-2A-E1B55K; and
with the vector pDC316-Survivin-E1B19K-2A-E1B55K as a template, amplifying the genes Survivin-E1B19K and Survivin-E1B19K-2A-E1B55K respectively, and inserting the amplified products into the vectors pDC316-EF1a-E1A and pDC316-hamsterTERT-E1A respectively by seamless cloning, to construct vectors named pDC316-EF1a-E1A-Survivin-E1B19K and pDC316-hamsterTERT-E1A-Survivin-E1B19K-2A-E1B55K, respectively;
S7. synthesizing an hTERT-recHA DNA, subjecting the hTERT-recHA DNA to PCR and inserting the PCR product into the vectors pDC316-EF1a-E1A-Survivin-E1B19K and pDC316-hamsterTERT-E1A-Survivin-E1B19K-2A-E1B55K by seamless cloning, to construct vectors named pDC316-EF1a-E1A-Survivin-E1B19K-hTERT-recH and pDC316-hamsterTERT-E1A-Survivin-E1B19K-2A-E1B55K-hTERT-recHA;
S8. PCR cloning a recHA DNA and inserting the recHA DNA downstream of CMV promoter in pDC316 vector to construct a non-replicating adenovirus recombinant vector named pDC316-CMV-recHA;
S9. co-transfecting the pDC316-CMV-recHA recombinant vector and a backbone plasmid (pBHGloxdeltaE1, 3Cre) into HEK293 cells (Low passage) to obtain a non-replicating recombinant adenovirus named Ad-JYT-CMV-recHA;
S10. co-transfecting the recombinant vectors pDC316-EF1a-E1A-Survivin-E1B19K-hTERT-recH and pDC316-hamsterTERT-E1A-Survivin-E1B19K-2A-E1B55K-hTERT-recHA respectively, and a backbone plasmid (pBHGloxdeltaE1, 3Cre), into HEK293 cells to obtain recombinant adenoviruses named Ad-EE1A-hTERT-recHA and Ad-HTE1A-hTERT-recHA, respectively; and
S11. subjecting the recombinant adenoviruses in steps S9 and S10 to plaque purification, then adding the purified recombinant adenoviruses to cultured HEK293 cells and 293Low passage cells, collecting 50 ml of a culture solution and cells after 72 hours, and taking a supernatant for later use; and adding a buffer solution to the cells, performing ultrasonic disruption, removing cell debris, then mixing the remaining material with the supernatant, adding 40% PEG8000 and NaCl for pelleting, collecting a pellet, then adding a buffer solution to prepare a suspension, performing chromatography using molecular sieve Sepharose 4FF, followed by ultrafiltration, centrifugation and concentration to obtain a purified recombinant adenovirus, and storing the purified recombinant adenovirus in a refrigerator at −80° C. for later use.

The specific procedure was as follows. For plaque purification, HEK293 cells and 293Low passage cells were cultured in 10 cm cell culture dishes. When the cells grew to a density of 70%, the recombinant adenovirus solution was added. The recombinant adenovirus solution was 10×serially diluted in a centrifuge tube from 10-1 to 10-7. 200 μl of each of the 10-5 to 10-7 virus solutions was taken, added to a cell culture dish containing 5 ml of a fresh culture medium, and gently mixed until uniform. Then the cell culture dish was placed in an incubator at 37° C. with 5% CO2, and cultured for 1 hour. After the virus solution was removed, the cells were washed with PBS, and a culture medium (containing 10% FBS, 1% penicillin and streptomycin) obtained by mixing 1% low melting point agarose with 2×DMEM culture medium at 1:1 was added. The cell culture dish was placed in an incubator at 37° C. with 5% CO2, and cultured for 10 days. Plaques were picked, with 10 plaques each time. A recombinant adenovirus with the highest HA expression was screened out by Western blot for the next experiment.

For virus purification, HEK293 cells and 293Low passage cells were cultured in 7.5 cm culture flasks. When the cells grew to a density of 70%, the recombinant adenovirus solution was added. After 72 hours, 50 ml of the culture solution with cells was collected and centrifuged at 2000 rpm for 10 minutes, and the supernatant was taken for later use. 5 ml of PBS buffer was added to the centrifuge tube containing the cells, and the cells were subjected to ultrasonic disruption and low-speed centrifugation to remove cell debris. The supernatant was taken and mixed with the cell supernatant. 40% PEG8000 and 2.5M NaCl were added for pelleting. After incubation for pelleting, the pellet was collected by centrifugation, and a buffer solution (10 mM Tris HCl, pH 8.0, 2 mM MgCl₂, 4% Sucrose) was added to prepare a suspension, and the suspension was subjected to chromatography with molecular sieve Sepharose 4FF, followed by ultrafiltration, centrifugation and concentration to obtain a purified recombinant adenovirus, which was stored in a refrigerator at −80° C. for later use.

By optimizing the nucleotide sequence of HA gene (excluding the portions which encode the transmembrane region and intracellular amino acids), the overexpression of HA protein is maximized as much as possible, and the recHA protein is overexpressed in mammalian cells and anchored on the cell surface. In addition, the transmembrane region and intracellular amino acids of wild-type H1N1 influenza virus HA protein are replaced with the transmembrane region of human epidermal growth factor receptor-2 (HER-2). For the full-length sequence of wild-type H1N1 human influenza virus HA gene, whether the nucleotide sequence is optimized or not, the transmembrane region and intracellular sequence cannot be artificially expressed in mammalian cells. Therefore, in the present disclosure, such an H1N1 human influenza virus HA gene is included, wherein after the nucleotide sequence is optimized, the transmembrane region and intracellular sequence of the wild-type HA gene are replaced with the transmembrane region of human epidermal growth factor receptor-2 (HER-2); or alternatively, after the nucleotide sequence is not optimized or partially optimized, the transmembrane region and intracellular sequence of the wild-type HA gene are replaced with the transmembrane region of another membrane protein, thereby enabling the recHA protein to be artificially expressed in mammalian cells, especially human cells, and to be anchored on the cell surface.

A non-replicating human adenovirus vector is used, and CMV promoter is used to control recHA glycoprotein gene, thereby enabling the recHA glycoprotein to be overexpressed in mammalian cells, especially human cells, and to be anchored on the cell surface. The novel adenovirus vector human influenza virus vaccine can achieve the effect of preventing influenza virus cold by nasal inoculation or intramuscular injection.

In the application of tumor treatment, with regard to the amino acid sequence of the influenza virus hemagglutinin glycoprotein, the transmembrane region and intracellular amino acids of wild-type HA protein are replaced with the transmembrane region of HER-2, and restrictive human TERT promoter is used to control recHA gene, thereby enabling the recHA glycoprotein to be selectively expressed in tumors and to be anchored on the tumor cell surface as a tumor-heterologous artificial antigen. Since the human TERT promoter perform well in lung cancer, breast cancer, colorectal cancer and hysteromyoma cells, the purpose of treating the above four tumors is achieved. For other types of tumors, other tumor-related promoters can be used to control recHA gene, such as promoters hamster TERT, Survivin, E2F, tyrosinase, prostate-specific antigen, alpha-fetoprotein and COX-2, and IRESs downstream of the promoters for the control of recHA gene, thereby enabling selective expression of recHA in different types of tumors so as to treat different types of tumors.

During the construction of Ad-EE1A-hTERT-recHA vector, EF1a promoter is used to control E1A gene. Since EF1a is a strong eukaryotic promoter, oncolytic adenovirus can replicate in tumors derived from epithelial cells, thus expanding the scope of tumor treatment and improving the oncolytic ability of the oncolytic adenovirus. Other promoters can also be used to control E1A gene, such as TK promoter and SV40 promoter, especially EF1a promoter, thereby enabling the recHA gene to be expressed and the recHA to be anchored on the cell surface when the virus replicates in different tumors, thus expanding the scope of tumor treatment with oncolytic adenovirus. In order to improve the safety of oncolytic adenovirus, Survivin promoter is used in the present disclosure to control E1B19K gene when constructing Ad-EE1A-hTERT-recHA vector, thereby improving the replication ability of the oncolytic adenovirus in tumors. E1B55K gene is deleted to realize selective replication of the oncolytic adenovirus in tumors, and reduce the replication ability of the oncolytic adenovirus in normal cells, thereby improving the safety of the oncolytic adenovirus. In order to expand the scope of tumor treatment, replacement can be made with tumor-related promoters, such as promoters hamster TERT, E2F, tyrosinase, prostate-specific antigen, alpha-fetoprotein and COX-2, and IRESs downstream of the promoters for the control of E1B19K gene, thus improving the replication ability of the oncolytic adenovirus in specific tumors.

During the construction of Ad-HTE1A-hTERT-recHA vector, hamster TERT promoter is used to control E1A gene, and Survivin promoter is used to control E1B19K and E1B55K fusion protein gene in which both are connected via 2A, thus improving the replication ability of the oncolytic adenovirus in specific tumors. In order to expand the scope of tumor treatment, replacement can be made with tumor-related promoters, such as promoters hamster TERT, E2F, tyrosinase, prostate-specific antigen, alpha-fetoprotein and COX-2, and IRESs downstream of the promoters for the control of E1B19K and E1B55K fusion protein gene in which both are connected via 2A, thus improving the replication ability of the oncolytic adenovirus in specific tumors. In order to limit the expression of the recHA antigen in different tumors, other tumor-related promoters can be selected, such as promoters hamster TERT, Survivin, E2F, tyrosinase, prostate-specific antigen, alpha-fetoprotein and COX-2, and IRESs downstream of the promoters for the control of E1A gene, such that tumor-related promoters are used to control recHA glycoprotein gene while the oncolytic adenovirus selectively replicates in tumors, and the recHA gene is expressed in different types of tumor cells and the recHA is anchored on the cell surface to achieve the purpose of treating different types of tumors. In addition, E3 gene is deleted to expand the insertion capacity of adenovirus, or the E3 gene may also be retained to enhance the oncolytic ability of the oncolytic adenovirus.

Restrictive hamster TERT promoter is used to control E1A gene, so as to enable the oncolytic adenovirus to selectively replicate in tumors and the influenza virus recHA glycoprotein to be limitedly expressed in tumors and to be anchored on the tumor cell surface, thus improving the safety of the oncolytic adenovirus. Since the nucleotide sequence of hamster TERT promoter is different from that of human TERT promoter with a low similarity, the probability of recombination between the two promoters is extremely low, which does not affect the normal replication of the recombinant oncolytic adenovirus in vivo and in vitro. Antibodies and memory B cells that have already existed in the body are utilized. Before clinical treatment, a patient is inoculated with a protein vaccine. After antibodies are produced in the patient, vectors carrying genes for corresponding proteins, such as plasmids, DNAs, and mRNAs, especially recombinant oncolytic viruses, are injected such that the corresponding proteins are expressed in tumor cells, whereupon the immune system accurately identifies and kills the tumor cells.

Adenovirus is epitheliophilic, and most human tumors are derived from epithelial cells. Therefore, this novel human influenza virus vaccine can treat tumors mainly derived from epithelial cells, including lung cancer, breast cancer, colorectal cancer, hysteromyoma, uterine cancer, prostate cancer, testicular cancer, pancreatic cancer, liver cancer, gastric cancer, renal cancer, bladder cancer, ovarian cancer, cervical cancer, head and neck cancer, brain cancer, esophageal cancer, melanoma, lymphoma, bone cancer, etc. The recombinant oncolytic virus carrying recHA gene may be directly injected into tumors or may also be administered by intravenous injection.

The recombinant vector may be human adenovirus type 5 or may also be other subtypes of adenovirus, herpes virus (lacking ICP34.5 and ICP47), poxvirus, measles virus, reovirus, Newcastle disease virus, parvovirus, Coxsackie virus, alphavirus or other oncolytic viruses, thereby enabling the human influenza virus recHA gene to be expressed in tumors and the recHA to be anchored on the cell surface.

Wild-type HA gene, whether optimized or not, is not expressed in HEK293 cells. When the nucleotide sequence of HA is optimized and the transmembrane region and intracellular sequence of the wild-type HA gene are replaced with the transmembrane region of HER-2, overexpression of the recHA glycoprotein in HEK293 cells and hamster DDT tumor cells is realized.

In order to artificially produce tumor-specific antigens and tumor-specific targets on the tumor cell surface to achieve the purpose of treating solid tumors, hemagglutinin (HA) glycoprotein of influenza A virus NYMCX-179A vaccine strain H1N1 recommended by WHO is selected as a tumor-heterologous antigen. After the nucleotide sequence of HA is optimized, the transmembrane region and intracellular sequence of the wild-type HA gene are replaced with the transmembrane region of HER-2, so that the recHA glycoprotein is overexpressed in tumors and anchored on the cell surface as a tumor-heterologous artificial antigen for treating the tumors. According to the principle of antibody-dependent cell-mediated cytotoxicity, the human influenza virus H1N1 hemagglutinin (HA) vaccine is used for improving the therapeutic effect on solid tumors. Anti-HA antibodies and memory B cells that have been produced in vivo are utilized. Before clinical treatment, a patient is injected with the H1N1 human influenza virus hemagglutinin (HA) vaccine such that anti-HA specific antibodies can be produced in the patient. After anti-HA antibodies exist in the patient, a recombinant oncolytic adenovirus carrying human influenza virus hemagglutinin (recHA) gene is injected. The recHA protein is expressed and anchored on the tumor cell surface as an artificial tumor-specific antigen while the oncolytic adenovirus replicates in the tumor cells. Since anti-HA specific antibodies have already existed in the patient, once hemagglutinin protein is expressed in the tumors and appears on the tumor cell surface, the immune system of the body is induced to identify the tumors and kill the tumor cells. A tumor killing experiment in animals showed that only two injections of recombinant oncolytic adenovirus Ad-EE1A-hTERT-recHA carrying recHA gene or three injections of recombinant oncolytic adenovirus Ad-HTE1A-hTERT-recHA could make 350 mm3 tumors disappear in 7-9 days, and achieved a cure rate of 100%.

The reasons for choosing the influenza virus H1N1 hemagglutinin (HA) protein as an antigen vaccine are as follows: firstly, human influenza virus vaccines are easy to obtain; secondly, the safety and effectiveness thereof have been confirmed in long-term applications; and thirdly, human influenza A (H1N1) viruses have been widely spread all over the world many times, and human beings have been universally inoculated with human influenza virus H1N1 vaccines, so that antibodies against H1N1 hemagglutinin (HA) and memory B cells are widespread in human bodies. Although anti-HA antibodies exist in the body for a limited period of time, since memory B cells can survive for a long period of time in the body (theoretically, they can survive for a human's lifetime), inoculation with an HA vaccine before treatment can make the body reproduce anti-HA antibodies within a short period of time. According to the tumor killing experiment in animals, it was shown that in the case of inoculating the animals with the HA protein vaccine before treatment, when the antibody titer in the animal reached 5000 EU, two injections of the recombinant oncolytic adenovirus carrying recHA gene (Ad-EE1A-hTERT-recHA) or three injections of the recombinant oncolytic adenovirus carrying recHA gene (Ad-HTE1A-hTERT-recHA) achieved a tumor killing rate of 100%. In hamsters in which no anti-HA antibodies were performed, two or three injections of the same recombinant oncolytic adenovirus (Ad-EE1A-hTERT-recHA or Ad-HTE1A-hTERT-recHA) against 350 mm3 tumors achieved a tumor killing rate of zero, and 100% of the experimental animals died, with the only difference being that the death time was delayed by about one month as compared with an untreated animal group. The time from the first injection of the oncolytic adenovirus to the complete cure of tumors was 7-9 days. On day 10, antibodies in the cured animals were detected, the antibody titer reached about 11000 EU, and the antibody level was more than doubled, indicating that memory B cells had already existed in the animals inoculated with the HA protein vaccine. The recombinant oncolytic adenovirus expressed recHA protein antigen in vivo, which induced an effect of strengthening immunity, thereby improving the anti-HA IgG level in the animals within a short period of time, a process beneficial for the immune system of the body to accurately identify and kill tumor cells.

Due to hydrophobic amino acid enrichment in the transmembrane region and intracellular region of the HA protein of the human influenza virus H1N1 strain, the full-length sequence of HA gene cannot be artificially expressed in mammalian cells and can only be expressed in insect cells. By optimizing the nucleotide sequence of the HA of the human influenza virus H1N1 strain and replacing the transmembrane region and intracellular sequence of the wild-type HA gene with the transmembrane region of HER-2, the HA protein was effectively expressed in vivo and in vitro and anchored on the cell surface as an influenza virus vaccine and a tumor-heterologous artificial antigen. After flow cytometry assay of HEK293 cells in vitro and tumor cells in animals, the recHA protein was successfully expressed and anchored on the cell surface. According to the experimental data that the antibody level in animals was more than doubled in 10 days, it was indicated that the recHA antigen on the tumor cell surface showed a stronger immunogenicity in vivo and effectively stimulated an immune response in the animals, thereby enabling the immune system to accurately identify and completely eliminate the tumor cells.

According to the principle that the optimization of the nucleotide sequence can improve the protein expression level, the nucleotide sequence of HA of NYMCX-179A (H1N1) vaccine strain is optimized in the present disclosure. The similarity between the optimized nucleotide sequence and the unoptimized HA sequence is 29.4%, which prevents the recombinant adenovirus carrying recHA gene from recombining with wild-type influenza virus HA in nature and improves the safety of the recombinant adenovirus of the present disclosure.

Human TERT promoter is used to control recHA protein gene, thereby enabling selective expression of the recHA protein antigen in lung cancer, breast cancer, colorectal cancer and hysteromyoma tumors, thus treating the above four tumors. The key to the remarkable effect thereof in the applications of treating tumors is to enable the HA glycoprotein of the human influenza virus H1N1 strain to be expressed in tumors and to be anchored on the cell surface, thereby forming an artificial heterologous specific antigen on the tumor cell surface. Before clinical treatment, the body is inoculated with an H1N1 HA vaccine, such that the body produces corresponding antibodies, and the antibodies in the body bind to the artificial heterologous antigen on the tumor cell surface, which improves the tumor killing effect. This new treatment method is suitable for treating almost all solid tumors and thus suitable for any glycoprotein vaccines for treating tumors, including viral glycoproteins, bacterial glycoproteins, and other proteins that can be prepared into vaccines, especially viral surface glycoproteins, such as influenza virus neuraminidase (NA), measles virus hemagglutinin (HA) and coronavirus spike protein(S).

Human beings acquire corresponding antibodies and memory B cells through natural virus infection or vaccination. The antibody titer in vivo varies depending on the antigen, and most antibodies decrease with time. Since memory B cells can survive for a long period of time in the body (theoretically, they can survive for a human's lifetime), inoculation with a corresponding antigen vaccine before treatment can make the body reproduce the corresponding antibodies within a short period of time. When an appropriate amount of antibodies have already existed in the patient, a vector that carries the corresponding gene by genetic engineering technology is injected into a tumor. Once the gene of interest is expressed in the tumor, glycoprotein appears on the tumor cell surface as a heterologous antigen. After the antibody binds to the heterologous antigen on the tumor surface, it activates a series of immune responses in the body, leading to the death of the tumor cells. The vector can be injected directly into the tumor, or may also be injected intravenously to make the vector reach the tumor area, for example, by transporting the gene of interest to the tumor area by means of lipid nanomaterials that encapsulate mRNAs, DNAs, plasmids, oncolytic viruses, etc.

Adenoviruses have no envelope glycoprotein and do not release viruses by means of budding. Since adenovirus E3A (gp19K) glycoprotein binds to the endoplasmic reticulum membrane of a cell, it basically does not appear on the cell surface, thus reducing the tumor-killing effect induced by the immune system of the body. By utilizing the fact that adenoviruses can infect most tumor cells, oncolytic adenoviruses carrying recHA glycoprotein gene can compensate for the deficiency that oncolytic adenoviruses cannot produce glycoproteins on the tumor surface. As the recHA glycoprotein, as an exogenous protein, does not exist in the structure of oncolytic adenoviruses, it does not affect the replication of the oncolytic adenoviruses in the body, thus improving the tumor-killing effect of the oncolytic adenoviruses.

A recombinant virus strain is provided, which is constructed by the above method.

The recombinant virus strain allows HA to be a tumor-specific artificial target, which is used in combination with a CART-HA cell for treating a tumor.

The constructed recombinant virus strain can be used for preparing a human influenza virus vaccine.

A novel human influenza virus vaccine is provided, wherein an antigen therein comprises the above recombinant virus strain.

Use of the novel human influenza virus vaccine in the field of treatment of influenza virus cold and tumors is provided.

### Example 2. Determination of Adenovirus Titer

The adenovirus titer was determined by a rapid immunoassay kit produced by Shenzhen Labkit Bioscience Co., Ltd. The adenovirus titer was determined by binding anti-adenovirus antibodies to cells infected with adenoviruses, then binding secondary antibodies labeled with horseradish peroxidase to the primary antibodies, and staining the infected cells with a chromogenic solution to develop a distinct brown color, followed by counting and calculation using the following formula.

The infectious unit per well (IFU)/ml was calculated:

$$( Average number of positive cells / visual field ) \times ( Number of visual fields / well ) / \{ Virus volume ( ml ) \times Dilution factor \}$$

### Example 3. Western Blot

(1) Sample preparation: HEK293 cells and 293 Low passage cells were passaged at a ratio of 1:3. The next day, the HEK293 Low passage cells were infected with the viruses Ad-JYT-CMV-HA and Ad-JYT-CMV-recHA, respectively, and the HEK293 cells were infected with Ad-EE1A-hTERT-recHA and Ad-HTE1A-hTERT-recHA. After 48 hours, the cells were collected and lysed on ice for 10-20 minutes by adding RIPA lysis buffer. A loading buffer was then added. The mixture was placed in a boiling water bath for 5 minutes and centrifuged at 12000×g for 5 minutes, and the supernatant was taken for loading.
(2) Electrophoresis: SDS-PAGE gel electrophoresis;
(3) Transfer to a membrane: Proteins were transferred to a PVDF membrane;
(4) Blocking: The membrane was incubated with 3% BSA at 37° C. for 1 hour;
(5) Incubation with primary antibodies: The blocking solution was removed, and an appropriate amount of a primary antibody reaction solution (HA primary antibodies dissolved in 1% BSA-TBST) was added at 0.2-0.3 ml/cm2; and the mixture was incubated at room temperature for 1 hour on a gently shaking shaker;
(6) Incubation with secondary antibodies: The primary antibody reaction solution was removed, and the PVDF membrane was washed with TBST three times, each time for 10 minutes, to remove unbound antibodies; and based on the membrane area, a secondary antibody reaction solution (HRP-labeled secondary antibodies dissolved in 1% BSA-TBST) was added at 0.1 ml/cm2, and the mixture was placed flat on a shaker and incubated at room temperature under shaking for 1 hour; and
(7) Development: The PVDF membrane was rinsed with TBST three times, each time for 10 minutes, to remove unbound antibodies; and the membrane was immersed in a freshly prepared ECL chemiluminescence reagent, developed and photographed on a chemiluminescence analyzer.

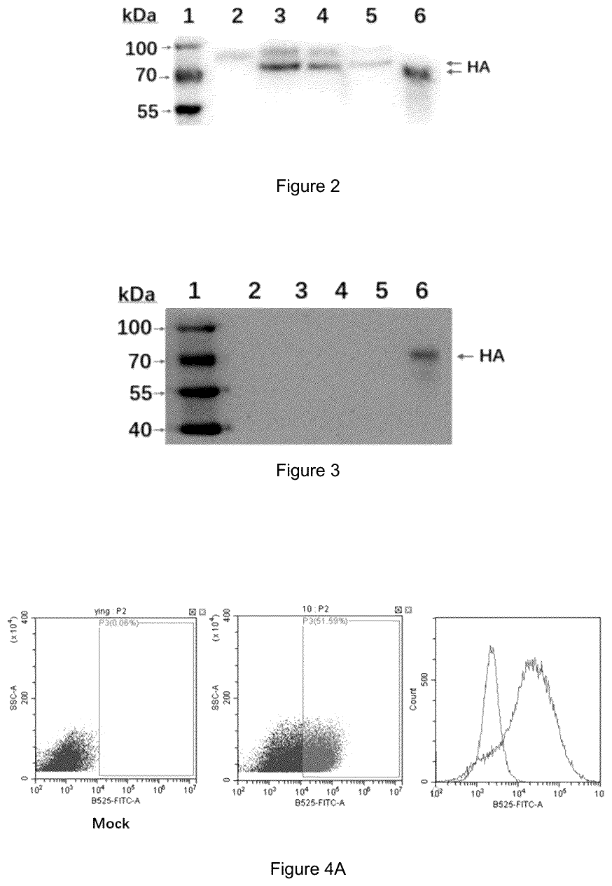

FIG. 2 shows the results of the Western blot assay, wherein (1) is marker; (2) is from recombinant oncolytic adenovirus Ad-JYT-CMV-HA (a non-replicating adenovirus, with CMV promoter controlling the full-length sequence of the wild-type HA gene); (3) is from infecting HEK293 Low passage cells with Ad-JYT-CMV-recHA (a non-replicating adenovirus, with CMV promoter controlling recHA gene); (4) is from infecting HEK293 cells with Ad-EE1A-hTERT-recHA and collecting the cells after 48 hours, and (5) is from infecting HEK293 cells with Ad-HTE1A-hTERT-recHA and collecting the cells after 48 hours; and (6) is purified hemagglutinin (HA) glycoprotein expressed by baculoviruses.

FIG. 3 shows the results of the Western blot assay, wherein (1) is marker; (2) is from recombinant adenovirus Ad-JYT-CMV-HA (a non-replicating adenovirus, with CMV promoter controlling the full-length sequence of the wild-type HA gene); (3) is from Ad-JYT-CMV-recHA (a non-replicating adenovirus, with CMV promoter controlling recHA gene); (4) is from Ad-EE1A-hTERT-recHA and (5) is from Ad-HTE1A-hTERT-recHA, which has been subjected to CsCl-based ultracentrifugation for virus purification; and (6) is purified hemagglutinin (HA) glycoprotein expressed by baculoviruses.

### Example 4. Flow Cytometry Assay In Vitro

Referring to FIG. 4A, HEK293 Low passage cells were cultured in a 10 cm culture flask. When the cells grew to a density of 70%, a recombinant adenovirus solution was added. After 24 hours, the cells were collected and centrifuged at 2000 rpm for 10 minutes. The supernatant was discarded, and 5 ml of PBS buffer was added to the centrifuge tube containing the cells to wash the cells. After the cells were fixed, the cells were washed with PBS buffer. The cells were reacted with a murine anti-HA monoclonal antibody (PBS containing 1% BSA) solution for 2 hours and reacted with FITC-labeled rabbit anti-murine IgG antibodies as secondary antibodies for 30 minutes, and detected by LX analytical flow cytometer (CytoFLEXLX).

### Example 5. Flow Cytometry Assay In Vivo

Referring to FIG. 4B, when the tumor grew to 600 mm3, a recombinant adenovirus was injected. A tumor tissue was taken 24 hours after a second injection of the recombinant adenovirus. The tumor tissue was cut into pieces with sterile surgical scissors. The tumor was treated with tumor digestion solution PBS (containing 1 mg/ml collagenase B, 0.1 mg/ml hyaluronidase, and 0.02 mg/ml DNase). Tumor cells from tumor-bearing hamsters were collected and suspended in 100 μl of PBS buffer. After fixation with 4% paraformaldehyde for 30 minutes, Fc was blocked with Fc Receptor Blocker (INNOVEX Biosciences Inc. California, USA). The cells were washed with PBS buffer and then reacted with a murine anti-HA monoclonal antibody (PBS containing 1% BSA) solution for 2 hours and reacted with FITC-labeled rabbit anti-murine IgG antibodies as secondary antibodies for 30 minutes. Tumor cells not injected with viruses were used as a negative control. Detection was performed using LX analytical flow cytometer (CytoFLEXLX).

### Example 6. Inoculation of Experimental Animals with Adenovirus Vector HA Vaccine

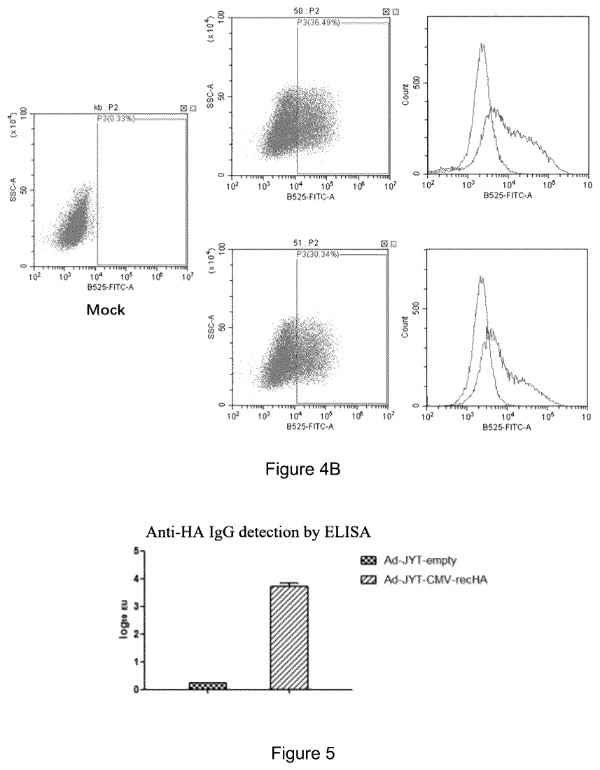

Referring to FIG. 5, 15 four-week-old female BALB/c mice were raised. 10 mice were inoculated with 50 μl of a 1×1010 Ad-JYT-CMV-recHA (PFU) PBS virus suspension by nasal instillation, and the control group was inoculated with 50 μl of a 1×1010 Ad-JYT-empty (PFU) PBS virus suspension. The inoculation was repeated for the second time on day 14. On day 28, orbital blood was taken to prepare sera, and the antibody concentration was detected by ELISA.

### Example 7: Inoculation of Experimental Animals with Hemagglutinin Protein Vaccines

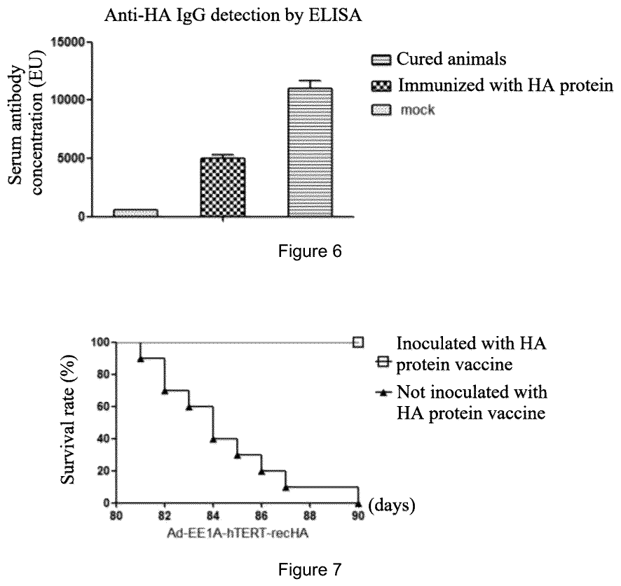

Referring to FIG. 6, 4-week-old Syrian hamsters were immunized with HA glycoprotein of influenza A virus NYMCX-157 (H1N1) strain as produced by a baculovirus expression vector (from Sino Biological Inc., Beijing). For the first injection, the prepared antigen was fully mixed with the same volume of Freund's complete adjuvant until uniform to prepare an injection, and each hamster was intraperitoneally injected with 200 μl of the injection (containing 10 μg of hemagglutinin protein). After injection, the needle remained in place for a few seconds to prevent the antigen from leaking out. After an interval of two weeks, the same antigen was emulsified with an equal volume of Freund's incomplete adjuvant and then administrated to the hamsters as a second injection for booster immunization. A total of two injections were given. On day 28, orbital blood was taken to prepare sera, and the antibody concentration was measured by ELISA.

### Example 8. Tumor Killing Experiment in Animals

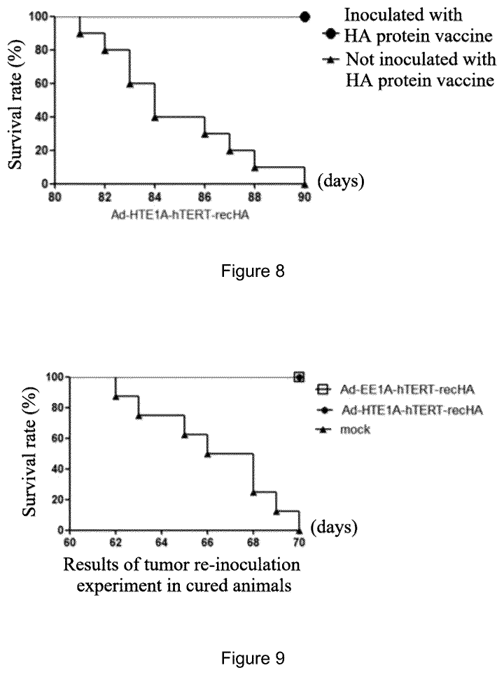

Referring to FIGS. 7 and 8, qualified hamsters (8 weeks old) inoculated with an H1N1 HA protein vaccine and tested positive for anti-HA antibodies by ELISA on day 28 were selected for the tumor killing experiment in animals, and meanwhile unvaccinated 8-week-old hamsters were used for a control tumor killing experiment. Syrian hamsters were inoculated with 100 μl of 5×106 DDT cells (hamster leiomyosarcoma cells) on the right back. The vaccinated hamsters were classified as one group, and the unvaccinated hamsters were classified as one group, with 10 hamsters in each group. When the tumors grew to 350 mm3, they were intratumorally injected with 100 μl of 1×109 PFU recombinant adenoviruses respectively, twice on days 0 and 2 or three times on days 0, 2 and 4. The recombinant adenovirus samples were Ad-EE1A-hTERT-recHA and Ad-HTE1A-hTERT-recHA. The health status of the experimental hamsters was observed every day, and the growth size of tumors and the survival number were recorded.

### Example 9. Experiment of Re-Inoculation of Cured Animals with Tumor Cells

Referring to FIG. 9, for the experiment of re-inoculation of cured animals with tumor cells, the hamsters cured with recombinant oncolytic adenoviruses Ad-EE1A-hTERT-recHA and Ad-HTE1A-hTERT-recHA were continued to be fed, and 8 hamsters were selected therefrom. After 70 days, the hamsters were further inoculated with 100 μl of 1×107 DDT cells on the right back. Eight 15-week-old healthy Syrian hamsters were selected as a control group and inoculated with 100 μl of 1×107 DDT cells on the right back. The growth of tumors and the survival number in each group were observed.

Some exemplary embodiments of the present disclosure have been described above by way of illustration only. Undoubtedly, those of ordinary skill in the art can modify the described embodiments in various ways without departing from the spirit and scope of the present disclosure. Therefore, the above drawings and descriptions are illustrative in nature and should not be construed as limiting the scope of protection of the claims of the present disclosure.

The invention claimed is:

1. A method for constructing a novel human influenza virus vaccine, specifically comprising the following steps:
S1. constructing recombinant HA (recHA) using a human influenza virus H1 N1 hemagglutinin glycoprotein gene, wherein the influenza virus hemagglutinin glycoprotein gene comprises amino acid sequences and optimized nucleotide sequences of HA1 and HA2, the recHA is constructed by replacing a transmembrane region and an intracellular sequence of a wild-type HA gene with a transmembrane region of human epidermal growth factor receptor-2 (HER-2), and the human influenza virus is vaccine strain H1 N1 NYMC X-179A recommended by WHO in which hemagglutinin glycoprotein acts as an antigen and includes HA glycoproteins of all human influenza virus subtypes;
S2. synthesizing a hamster Enhancer TERT DNA fragment, the 5′ end and 3′ end of which are provided with restriction sites Xbal and EcoRI respectively, digesting shuttle vector pDC316 and the hamster Enhancer TERT DNA with double enzymes, Xbal and EcoRI, respectively, followed by recovery, and inserting the synthesized Hamster Enhancer TERT DNA fragment at the Xbal and EcoRI sites for enzymatic linkage to construct a vector named pDC316-hamsterTERT;
S3. synthesizing an EF1 a DNA fragment, the 5′ end and 3′ end of which are provided with restriction sites Xbal and EcoRI respectively, digesting shuttle vector pDC316 and EF1 a DNA with double enzymes, Xbal and EcoRI, respectively, followed by recovery, and inserting the synthesized EF1 a DNA fragment at the Xbal and EcoRI sites for enzymatic linkage to construct a vector named pDC316-EF1 a;
S4. synthesizing EIA gene, the 5′ end and 3′ end of which are provided with restriction sites EcoRI and BgIII respectively, digesting vector pDC316-hamsterTERT and the E1A gene with double enzymes, EcoRI and BgIII, respectively, followed by recovery and linkage to construct a vector named pDC316-hamsterTERT-E1 A, subjecting the EIA gene and vector pDC316-EF1 a to PCR, recovering the PCR products respectively, performing seamless cloning to construct a vector named pDC316-EF1 a-E1 A, and identifying the correctness of DNA insertion into the recombinant vector by sequencing;
S5. synthesizing a Survivin fragment, the 5′ end and 3′ end of which are provided with restriction sites Xbal and EcoRI respectively, digesting shuttle vector pDC316 and Survivin with double enzymes, Xbal and EcoRI, respectively, followed by recovery, and inserting the synthesized Survivin fragment at the Xbal and EcoRI sites for enzymatic linkage to construct a vector named pDC316-Survivin;
S6. synthesizing E1 B19K-2A-E1 B55K gene, the 5′ end and 3′ end of which are provided with restriction sites EcoRI and Sall respectively, digesting vector pDC316-Survivin and the E1 B19K-2A-E1 B55 gene with double enzymes, EcoRI and Sall, respectively, followed by recovery, and inserting the synthesized E1 B19K-2A-E1 B55K gene at the EcoRI and Sall sites for enzymatic linkage to construct a vector named pDC316-Survivin-E1 B19K-2A-E1 B55K; and
with the vector pDC316-Survivin-E1 B19K-2A-E1 B55K as a template, amplifying the genes Survivin-E1 B19K and Survivin-E1 B19K-2A-E1 B55K respectively, and inserting the amplified products into the vectors pDC316-EF1 a-E1 A and pDC316-hamsterTERT-E1 A respectively by seamless cloning, to construct vectors named pDC316-EF1 a-E1 A-Survivin-E1 B19K and pDC316-hamsterTERT-E1 A-Survivin-E1 B19K-2A-E1 B55K, respectively;
S7. synthesizing an hTERT-recHA DNA, inserting the hTERT-recHA DNA into the vectors pDC316-EF1 a-E1A-Survivin-E1 B19K and pDC316-hamsterTERT-E1 A-Survivin-E1 B19K-2A-E1 B55K by seamless cloning, to construct vectors named pDC316-EF1 a-E1 A-Survivin-E1 B19K-hTERT-recHA and pDC316-hamsterTERT-E1 A-Survivin-E1 B19K-2A-E1 B55K-hTERT-recHA, respectively;
S8. cloning a recHA DNA and inserting the recHA DNA downstream of CMV promoter in pDC316 vector to construct a non-replicating adenovirus recombinant vector named pDC316-CMV-recHA;
S9. co-transfecting the pDC316-CMV-recHA recombinant vector and a backbone plasmid into HEK293 cells (Low passage) to obtain a non-replicating recombinant adenovirus named Ad-JYT-CMV-recHA;
S10. co-transfecting the recombinant vectors pDC316-EF1 a-E1A-Survivin-E1 B19K-hTERT-recH and pDC316-hamsterTERT-E1A-Survivin-E1 B19K-2A-E1 B55K-hTERT-recHA respectively, and a backbone plasmid, into HEK293 cells to obtain recombinant adenoviruses named Ad-EE1A-hTERT-recHA and Ad-HTE1A-hTERT-recHA, respectively; and
S11. subjecting the recombinant adenoviruses in steps S9 and S10 to plaque purification, then adding the purified recombinant adenoviruses to cultured HEK293 cells and 293 Low passage cells, collecting 50 ml of a culture solution and cells after 72 hours, and taking a supernatant for later use; and adding a buffer solution to the cells, performing ultrasonic disruption, removing cell debris, then mixing the remaining material with the supernatant, adding 40% PEG8000 and NaCl for pelleting, collecting a pellet, then adding a buffer solution to prepare a suspension, performing chromatography using molecular sieve Sepharose 4FF, followed by ultrafiltration, centrifugation and concentration to obtain a purified recombinant adenovirus, and storing the purified recombinant adenovirus in a refrigerator at −80° C. for later use.

2. A recombinant virus strain, wherein the recombinant virus strain is constructed by the method according to claim 1.

3. The recombinant virus strain according to claim 2, wherein the recombinant virus strain allows HA to be a tumor-specific artificial target, which is used in combination with a CART-HA cell for treating a tumor.

4. Use of the recombinant virus strain according to claim 2 in the preparation of a human influenza virus vaccine.

5. A novel human influenza virus vaccine, wherein an antigen in the novel human influenza virus vaccine comprises the recombinant virus strain according to claim 2.

6. Use of the novel human influenza virus vaccine according to claim 4 in the field of treatment of influenza virus cold and tumors.
